(19) United States
(12) Patent Application Publication
(10) Pub. No.: US 2013/0310690 A1
(43) Pub. Date: Nov. 21, 2013

(54) BREAST ULTRASOUND SCANNING AND DIAGNOSIS AID SYSTEM

(71) Applicants: Ruey-Feng Chang, Taipei (TW); Yi-Hong Chou, Taipei (TW); Chiun-Sheng Huang, Taipei (TW); Yeun-Chung Chang, Taipei (TW); Shao-Chien Chang, Taipei (TW); Min-Chun Yang, Taipei (TW); Yao-Sian Huang, Taipei (TW); Chung-Ming Lo, Taipei (TW)

(72) Inventors: Ruey-Feng Chang, Taipei (TW); Yi-Hong Chou, Taipei (TW); Chiun-Sheng Huang, Taipei (TW); Yeun-Chung Chang, Taipei (TW); Shao-Chien Chang, Taipei (TW); Min-Chun Yang, Taipei (TW); Yao-Sian Huang, Taipei (TW); Chung-Ming Lo, Taipei (TW)

(73) Assignee: NATIONAL TAIWAN UNIVERSITY, Taipei (TW)

(21) Appl. No.: 13/585,969

(22) Filed: Aug. 15, 2012

(30) Foreign Application Priority Data
May 18, 2012 (TW) 101117704

(51) Int. Cl. A61B 8/08; A61B 8/13

(52) U.S. Cl. 600/443

(57) ABSTRACT

Provided herein is a breast ultrasound scanning and diagnosis aid system. The scanning aid system is capable of aiding positioning and tracking the scanned breast ultrasound images and suspicious tumor ultrasound images so that the doctor can be easily informed of the scanned positions of the breast ultrasound images and the suspicious tumor ultrasound images. The diagnosis aid system selects at least one representative, breast ultrasound images with tumor characteristics, segments a suspicious tumor region, and acquires a plurality of characteristic parameters in association with tumor tissues from the suspicious tumor region so as to provide a diagnostic suggestion. With the use of the present invention, the positions of suspicious tumors can be acquired from the positioning data of the breast ultrasound images, and tumor diagnosis accuracy can be improved by selecting an ultrasound image with tumor characteristics.

### FIELD OF THE INVENTION

The present invention generally relates to a breast ultrasound scanning and diagnosis aid system and, more particularly, to a system capable of aiding scanning and diagnosing breast ultrasound images.

### BACKGROUND OF THE INVENTION

Breast cancer has become one of the commonest cancers for women. According to clinical experiences, the cure rate would be rather high if a patient is medically treated right alter being diagnosed with breast cancer at an early stage.

Currently, the ultrasound breast examination is one of major techniques for breast lesion checkup because of its advantages such as non-radioactivity, non-invasiveness, harmlessness to breast tissues, accurate positioning, convenience and ease of use. Most of all, it is less costly compared to CT and MRT. Moreover, ultrasound breast examination is suitable for Asian women because their breast tissues are denser.

Although breast ultrasound is a common medical checkup, there are still some problems to be solved when such checkup is carried out.

First, the conventional ultrasound breast examination is performed manually by a doctor who grabs, with one hand, a scanner with a probe to scan back and forth on breasts while records, with the other, data such as the position of each tumor, the probe direction and the measured maximum tumor dimension that are inputted through an input device such as a keyboard. It may take plenty of time to complete the ultrasound breast examination because the recording process is also time-consuming. Moreover, the data such as the position each tumor, the probe direction and the measured maximum tumor dimension are acquired by a doctor who estimates by the eye, which may affect the accuracy of the acquired data.

Second, the image acquired during breast ultrasound scanning is an instant image. Some parts of the breasts may be left unscanned because the doctor has to look at the screen and record the data at the same time.

Third, plenty of breast ultrasound images are acquired during breast ultrasound. Afterwards, the doctor has to select at least one image with best tumor characteristics from these breast ultrasound images. However, the image selection process may take plenty of time and the quality of selected images may vary according to the experience and opinion of the doctor.

Accordingly, the present invention provides a system capable of aiding the doctor to scan and diagnose the breast ultrasound images. With the use of the breast ultrasound scanning and diagnosis aid system, the time for breast ultrasound examination can be shortened, the quality of breast ultrasound can be controlled, and initial diagnosis of breast ultrasound images can be achieved.

### SUMMARY OF THE INVENTION

It is one object of the present invention to provide a breast ultrasound scanning aid system, wherein the ultrasound images as well as positioning information such as the scanning position and scanning angle are recorded during breast ultrasound scanning so that the doctor is able to acquire the scanning position of the ultrasound images accurately and select the ultrasound images according to the positioning information.

It is another object of the present invention to provide a breast ultrasound scanning aid system, wherein the probe position, the probe rotation angle and the tumor position are automatically recorded and the maximum tumor dimension is automatically estimated so that the time for inputting data may be shortened during ultrasound breast examination.

It is still another object of the present invention to provide a breast ultrasound scanning aid system, which provides a scanning failure alert and a suspicious tumor alert so as to remind the doctor of scanning failures and suspicious tumors.

It is still another object of the present invention to provide a breast ultrasound diagnosis aid system, which is capable of automatically selecting at least one high quality representative ultrasound image with tumor characteristics so that the time for the doctor to select the suitable ultrasound images for diagnosis can be shortened and the diverseness image quality due to the experiences and opinions of the doctors will be eliminated.

It is still another object of the present invention to provide a breast ultrasound diagnosis aid system, which is capable of providing a plurality of control points on the boundaries of the suspicious tumors, the boundaries of the suspicious tumors can be re-defined by the adjustment of the control points so as to provide with the suitable tumor boundaries according to the doctor's demand.

To achieve above objects, the present invention provides a breast ultrasound scanning aid system, comprising: a positioning module for positioning a nipple, calculating a probe position, a probe rotation angle and/or at least one tumor position with the nipple as a center, and estimating at least one maximum tumor dimension; an image acquisition module for acquiring a plurality of breast ultrasound images; a continuous whole breast image positioning, and storage module for recording all the breast ultrasound images as well as scanning positions and scanning angles thereof; and a tumor image positioning and storage module for recording at least one suspicious tumor ultrasound image as well as a scanning position and a scanning angles thereof, the tumor position and the maximum tumor dimension.

In one embodiment of the present invention, the breast ultrasound scanning aid system further comprising a scanning trajectory module for recording a probe scanning trajectory and projecting the probe scanning trajectory onto a scanning trajectory map.

In one embodiment of the present invention, the breast ultrasound scanning aid system further comprising a scanning failure alert module for analyzing the continuity of the probe scanning, trajectory to determine whether to issue a scanning failure alert or not.

In one embodiment of the present invention, the breast ultrasound scanning aid system further comprising a suspicious tumor alert module for issuing a suspicious tumor alert after identifying the at least one suspicious tumor ultrasound image from the breast ultrasound images.

In one embodiment of the present invention, wherein the suspicious tumor alert module identifies at least one breast ultrasound image with a darker region from the breast ultrasound images and determines the at least one breast ultrasound image with the draker region as the suspicious tumor ultrasound image and the darker region as a suspicious tumor region.

In one embodiment of the present invention, wherein the positioning module performs positioning magnetically or optically.

The present invention further provides a breast ultrasound diagnosis aid system, comprising: a representative image selection module for selecting at least one representative breast ultrasound image with tumor characteristics from a plurality of breast ultrasound images; a tumor contour segmentation module for segmenting a suspicious tumor region from the representative breast ultrasound image; and a computer-aided diagnosis module for acquiring a plurality of characteristic parameters in association with tumor tissues from the suspicious tumor region and providing a diagnostic suggestion after analyzing the characteristic parameters.

In one embodiment of the present invention, the breast ultrasound diagnosis aid system further comprising an ultrasound mode analysis module for analyzing an ultrasound mode used in each of the breast ultrasound images.

In one embodiment of the present invention, wherein the ultrasound mode is a B-mode, a Doppler mode or an elastography mode.

In one embodiment of the present invention, the breast ultrasound diagnosis aid system further comprising an image selection module for providing a menu interlace that displays each of the ultrasound mode used in each of the breast ultrasound images, wherein the breast ultrasound image with a specific ultrasound mode can be viewed through clicking the menu interface.

In one embodiment of the present invention, wherein the representative image selection module for sorting the breast ultrasound images according to respective ratio values of darker regions or regions with harder elasticity in each of the breast ultrasound images and then selecting the representative breast ultrasound image from the sorted breast ultrasound images according, to at least one image selection number acquired by multiplying the number of the breast ultrasound images with at least one image selection parameter.

In one embodiment of the present invention, wherein the representative image selection module is provided with at least one color threshold value so that regions on the breast ultrasound images with average pixel values higher than the color threshold value are classified as lighter regions or regions with softer elasticity while regions on the breast ultrasound images with average pixel values lower than the color threshold value are classified as darker regions or regions with harder elasticity.

In one embodiment of the present invention, wherein the elasticity of the suspicious tumor region is classified by a fuzzy c-means algorithm.

In one embodiment of the present invention, wherein the tumor contour segmentation module identifies a tumor boundary of at least one suspicious tumor region from the representative breast ultrasound image, provides a plurality of control points on the tumor boundary of the suspicious tumor region so that the tumor contour segmentation module creates an re-formed tumor boundary of the suspicious tumor region by adjusting the control points.

In one embodiment of the present invention, the breast ultrasound diagnosis aid system further comprises a diagnosis report generating module for receiving the diagnostic suggestion from the computer-aided diagnosis module so as to generate a breast diagnosis report.

### BRIEF DESCRIPTION OF THE DRAWINGS

The objects and spirits of the embodiments of the present invention will be readily understood by the accompanying drawings and detailed descriptions, wherein:

FIG. 1 is a system structure chart of a breast ultrasound scanning aid system according to one embodiment of the present Invention;

FIG. 2 is a schematic diagram showing the positioning of a tumor with the nipple as a center according to one embodiment of the present invention;

FIG. 3 is a schematic diagram showing the maximum tumor dimension according to one embodiment of the present invention;

FIG. 4 shows the scanning trajectory of the probe according to the present invention;

FIG. 5 is a system structure chart of a breast ultrasound diagnosis aid system according to one preferred embodiment of the present invention;

FIG. 6A and FIG. 6B are schematic diagrams showing a plurality of control points on the boundary of a tumor region for re-defining the boundary by adjusting the control points according to one embodiment of the present invention; and

FIG. 7 is a schematic diagram of a menu interface of an image selection module according to one embodiment of the present invention.

### DETAILED DESCRIPTION OF THE PREFERRED EMBODIMENTS

The present invention may be exemplified but not limited by various embodiments as described hereinafter.

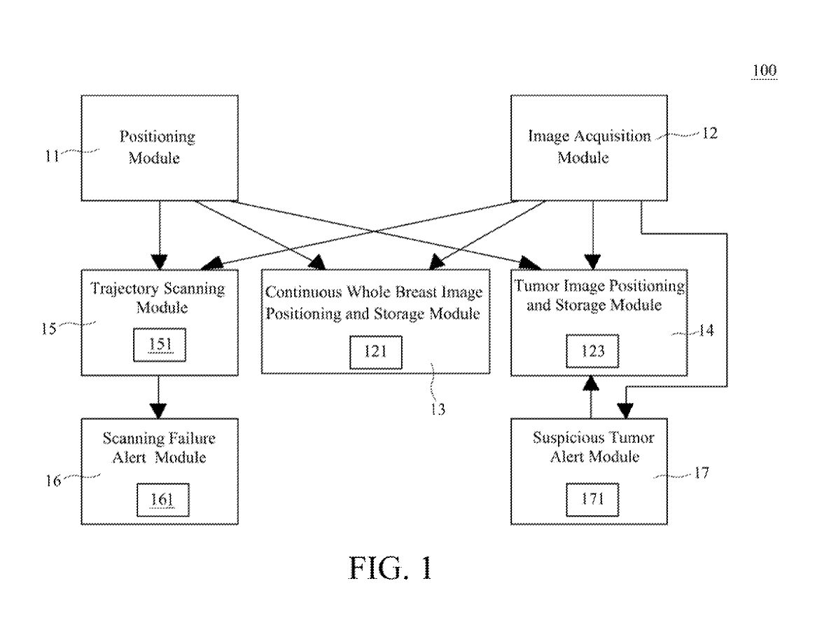

Please refer to FIG. 1, there is shown a system structure chart of a breast ultrasound scanning aid system according to one preferred embodiment of the present invention. As shown in the figure, the breast ultrasound scanning aid system 100 comprises a positioning module 11, an image acquisition module 12, a continuous whole breast image positioning and storage module 13 and a tumor image positioning and storage module 14.

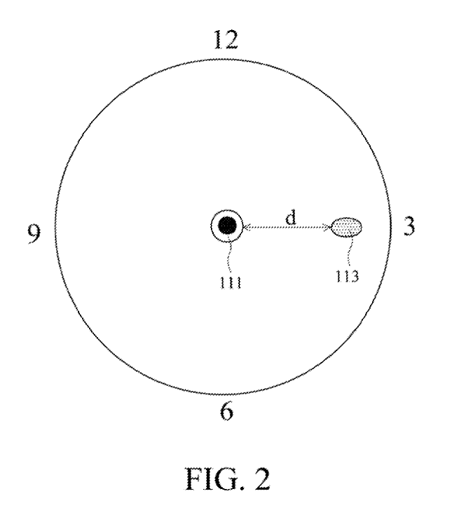

The positioning module 11 is installed on an ultrasound probe to perform positioning magnetically or optically. The positioning module 11 is a 6 degrees of freedom (such as x-y-z position and α, β, γ angle) positioning module. Moreover, before performing a breast ultrasound scanning process, the positioning module 1 of the scanning aid system 100 positions a nipple 111 and defines a 12 o'clock direction according to the position of the nipple 111, as shown in FIG. 2. The probe performs the breast ultrasound scanning process on the breasts so as to acquire continuous breast ultrasound images 121 by the image acquisition module 12. Furthermore, while the probe is scanning, the positioning module 11 calculates the probe position (such as direction and distance) as well as the probe rotation angle. Afterwards, each of the breast ultrasound images 121 and the corresponding scanning position and scanning angle will be stored in the continuous whole breast image positioning and storage module 13.

During ultrasound scanning, the doctor is able to find at least one suspicious tumor ultrasound image 123 and performs selected operation. Meanwhile, the positioning module 11 also positions the suspicious tumor ultrasound image 123 so that the suspicious tumor ultrasound image 123 selected by the doctor and the corresponding scanning position and scanning angle may be stored in the tumor image positioning and storage module 14.

Thereby, during ultrasound scanning, in addition to the ultrasound images 121/123, positioning data such as the scanning position and the scanning rotation angle are also recorded so that the doctor is able to acquire the scanning position of the ultrasound images 121/123 accurately and select the ultrasound images 121/123 according to the positioning data.

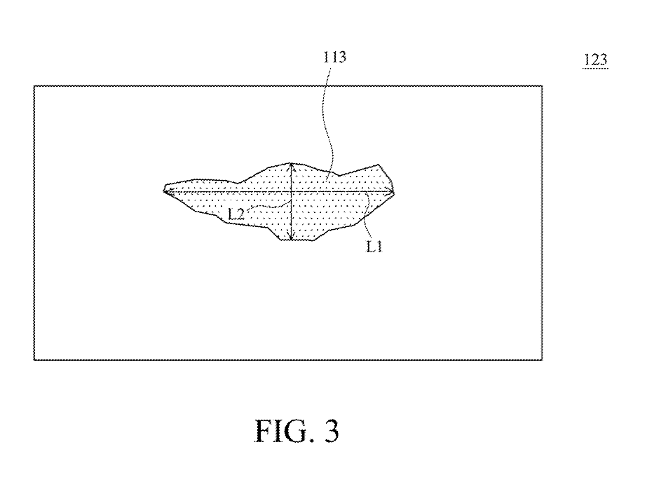

The positioning, module 11 calculates the position of the suspicious tumor with the nipple 111 as a center after identifying the suspicious tumor. The tumor position is expressed by means of clock directions and distances. Take FIG. 2 as an example, the position of the suspicious tumor 113 can be calculated at 3 o'clock direction of nipple 111 and 5 cm away from the nipple 111. Moreover, the positioning module 11 of the present invention can further estimates the maximum dimension (L1, L2) of the suspicious tumor 113 with the use of an image algorithm, as shown in FIG. 3. The position and the maximum dimension (L1, L2) of the suspicious tumor 113 are automatically recorded in the tumor image positioning and storage module 14 so that the doctor will be able to find the position of the same tumor 113 and be informed of the maximum dimension (L1, L2) of the tumor 113 in the future.

Accordingly, the breast ultrasound scanning aid system 100 of the present invention automatically records the probe position, the probe rotation angle and the position of the tumor 113, and automatically estimates the maximum tumor dimension. Thereby, the time for inputting data can be shortened during the breast ultrasound examination.

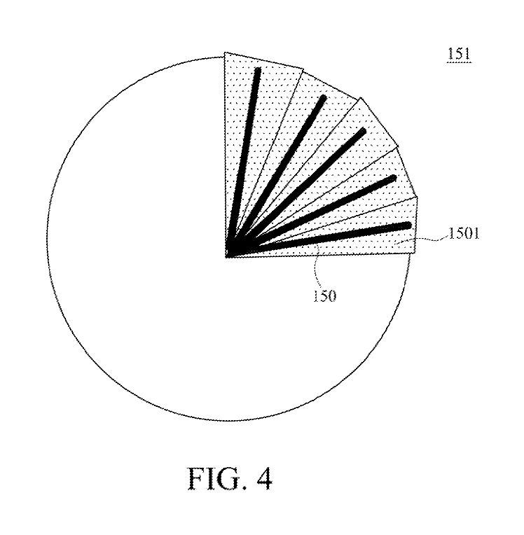

The scanning aid system 100 further comprises a scanning trajectory module 15 and a scanning failure alert module 16. During the breast ultrasound examination, the scanning trajectory module 15 records the continuous movement of probe. Furthermore, in order to inform the doctor about the scanning position, the scanning trajectory module 15 projects the scanning trajectory 150 of the probe onto a scanning trajectory map 151 and displays the scanned regions 1501 on the scanning trajectory map 151 according to the width of the probe, as shown in FIG. 4. The scanning failure alert module 16 is used for analyzing the trajectory continuity of the scanning trajectory 150. For example, the continuous variation of the angle, the position and the image of the probe are analyzed to determine whether the scanning, trajectory 150 continuously slow moves so as to determine whether to issue a scanning failure alert 161 or not. Herein, with the alert from the scanning failure alert module 16, the doctor can be prevented from leaving some parts of the breasts unscanned due to the probe is moving quickly during the ultrasound scanning.

Moreover, in one embodiment of the present invention, the suspicious tumor ultrasound image 123 can be selected from the continuously acquired breast ultrasound images 121 by the eye according to the experience of the doctor. Alternatively, in another embodiment of the present invention, the suspicious tumor ultrasound image 123 can be identified from the continuously acquired breast ultrasound images 121 by an automatical detection that is executed by a suspicious tumor alert module 17 of the scanning aid system 100.

For general ultrasound scanning, the grey scale value of a tumor tissue is darker compared to that of normal tissue. Therefore, in the present invention, the suspicious tumor alert module 17 identifies at least one breast ultrasound image with a darker region (with lower grey scale values) from the continuous breast ultrasound images 121, and determines the breast ultrasound image 121 with the darker region as a suspicious tumor ultrasound image 123 and the darker region as a suspicious tumor region. Moreover, the suspicious tumor alert module 17 issues a suspicious tumor alert 171 to remind the doctor of the suspicious tumor after identifying the suspicious tumor ultrasound image 123.

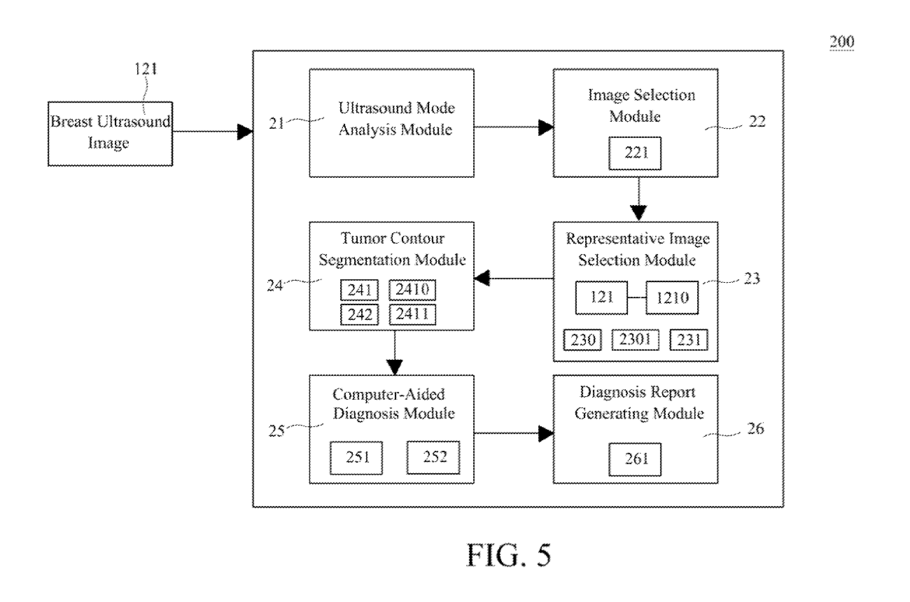

Please refer to FIG. 5, there is shown a system structure chart of a breast ultrasound diagnosis aid system according to one preferred embodiment of the present invention. As shown in the figure, the breast ultrasound diagnosis aid system 200 capable of receiving a plurality of breast ultrasound images 121 scanned by the breast ultrasound scanning aid system 100 comprises a representative image selection module 23, a tumor contour segmentation module 24 and a computer-aided diagnosis module 25.

Wherein the representative image selection module 23 is capable of selecting at least one representative breast Ultrasound image 231 with tumor characteristics from a plurality of breast ultrasound images 121. The representative image selection method as described following:

First, the representative image selection module 23 calculates the occupied percentage of darker regions (with lower grey scale values) or regions with harder elasticity (which are often presented by colors with lower grey scale values, blue or red for being distinguished from regions with softer elasticity) in each of the breast ultrasound images 121 to further acquire a ratio value 1210. Moreover, the total number (T) of the breast ultrasound images 121 is multiplied with at least one image selection parameter (S) 230 to acquire at least one image selection code (N) 2301, which is expressed as N=T×S, wherein the image selection parameter (S) 230 is a fraction smaller than 1.

After acquiring the ratio value 1210 corresponding to each of the breast ultrasound images 121, the representative image selection module 23 sorts the breast ultrasound images 121 according to respective ratio values 1210. Afterwards, the representative image selection module 23 selects at least one representative breast ultrasound image 231 from the sorted breast ultrasound images 121 according to the image selection code 2301.

For example, if the total number (T) of the breast ultrasound images 121 is 100, the image selection parameter (S) 230 is ¼, we obtain an image selection code (N) 2310 that is N=T>S=25. Accordingly, the representative image selection module 23 selects the 25th breast ultrasound image 121 from the sorted breast ultrasound images 121 as the representative breast ultrasound image 231.

In another embodiment of the present invention, the representative image selection module 23 can be provided with a plurality of image selection parameters (S) 230, such as S=¼, 2/4, ¾, to obtain a plurality of representative breast ultrasound images 231, such as the breast ultrasound images 121 numbered the 25th, the 50th and the 75th.

Moreover, in the present invention, the image selection code 2301 may not be an integer. Then, only the integral part is taken as the image selection code 2301 For example, if T=70 and S=¼, we obtain In this case, only the integral part, N=17, is taken as the image selection code 2301.

Moreover, the representative, image selection module 23 can also be provided with at least one color threshold value 232. Regions on the breast ultrasound images 121 with average pixel values (such as the average grey scale value or the average chroma value) higher than the color threshold value 232 are classified as lighter regions or regions with softer elasticity while regions on the breast ultrasound images 121 with average pixel values lower than the color threshold value 232 are classified as darker regions or regions with harder elasticity.

Accordingly, the diagnosis aid system 200 of the present invention can automatically selects at least one high-quality representative breast ultrasound images 231 with tumor characteristics. Therefore, the time for the doctor to select suitable ultrasound images for diagnosis can be shortened, and the diverseness in image quality due to the experiences and opinions of the doctors will be eliminated.

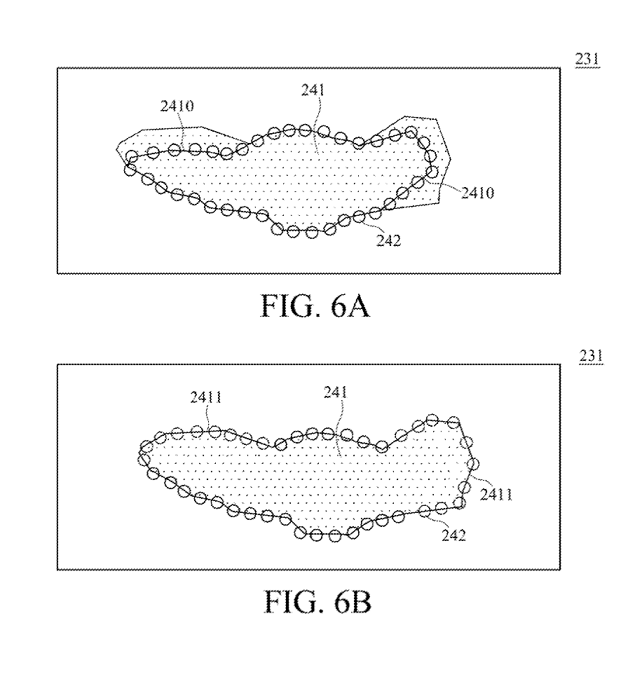

After acquiring at least one representative breast ultrasound image 231, as shown in FIG. 6A, the tumor contour segmentation module 24 identifies a tumor boundary 2410 of at least one suspicious tumor region 241 from the representative breast ultrasound image 231 and provides a plurality of control points 242 adjusted by the user on the tumor boundary 2410 of the suspicious tumor region 241 so that the tumor boundary 2410 can be corrected by adjusting the control points 242. If the tumor boundary 2410 identified by the tumor contour segmentation module 24 fails to meet the doctor's requirement, as shown in FIG. 6B, the number and the positions of the control points 242 can be re-defined by an operation device (such as a mouse) so that the tumor contour segmentation module 24 can create a re-formed tumor boundary 2411. Afterwards, the tumor contour segmentation module 24 can provide with a new suspicious tumor region 241 by the re-formed tumor bounding 2411 so as to meet the doctor's requirement.

After the suspicious tumor region 241 is segmented, the computer-aided diagnosis module 25 acquires a plurality of characteristic parameters 251 in association with tumor tissues (such as the volume, the average grey scale value, the standard deviation of the grey scale value, the grey scale difference between neighboring tissues, etc) from the suspicious tumor region 241, and provides a diagnostic suggestion 252 after analyzing the characteristic parameters 251. Accordingly, the diagnostic suggestion 252 reveals some information such as the shape of the tumor, the continuity of the tumor boundary, the elasticity score of the tumor and/or the grading of the tumor based on Breast Imaging-Reporting and Data System (BI-RADS) in the suspicious tumor region 241 for the doctor's reference.

Moreover, in one embodiment of the present invention, the elasticity of the suspicious tumor region 241 is classified by a fuzzy c-means algorithm.

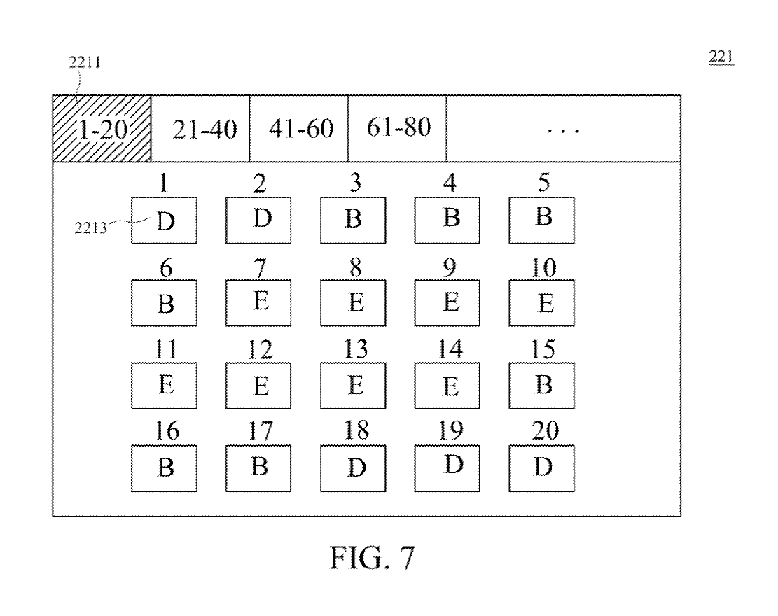

Furthermore, the diagnosis aid system 200 of the present invention further comprises an ultrasound mode analysis module 21 and an image selection module 22. The ultrasound mode analysis module 21 is used for analyzing the ultrasound mode used in each of the breast ultrasound images, such as a B-mode, a Doppler mode or an elastography mode. The image selection module 22 provides a menu interface 221. The menu interface 221 displays each of the ultrasound mode used in each of the breast ultrasound images 121 via the form of icons or texts. The doctor can view the breast ultrasound images 121 by clicking the menu interface 221. As shown in FIG. 7, the menu interface 221 displays a plurality of sub-menus 2211, each of sub-menus 2211 comprising, a plurality of options 2213. Each of the options 2213 corresponds to a breast ultrasound image 121 and is labeled an ultrasound mode used in the corresponding breast ultrasound image 121. For example, “B” stands for the B-mode, “D” stands for the Doppler mode, and “E” stands for the elastography mode.

Thereby, the doctor can select a breast ultrasound image 121 with a suitable ultrasound mode through clicking the options 2213 on the menu interface 221 for early-stage diagnosis. Moreover, the representative image selection module 23 can select the representative breast ultrasound image 231 from the breast ultrasound images 121 with the specific ultrasound mode, such as the elastography mode.

Finally, the doctor inputs the diagnostic suggestion 252 provided by the computer-aided diagnosis module 25 and/or the diagnostic opinion from the doctor itself into a diagnosis report generating module 26 so as to generate a breast diagnosis report 261.

Accordingly, with the use of the present invention, the scanned positions in the breast ultrasound images as well as the positions of the suspicious tumors can be acquired from the positioning data, and an ultrasound image with tumor characteristics can be automatically selected so as to define a suspicious tumor region, accurately. Thereby, the accuracy of tumor diagnosis can be improved to achieve quality control of breast ultrasound.

Although this invention has been disclosed and illustrated with reference to particular embodiments, the principles involved are susceptible for use in numerous other embodiments that will be apparent to persons skilled in the art. This invention is, therefore, to be limited only as indicated by the scope of the appended claims.

What is claimed is:

1. A breast ultrasound scanning aid system, comprising:
a positioning module for positioning a nipple, calculating, a probe position, a probe rotation angle and/or at least one tumor position with the nipple as a center, and estimating at least one maximum tumor dimension;
an image acquisition module for acquiring a plurality of breast ultrasound images;
a continuous whole breast image positioning and storage module for recording all the breast ultrasound images as well as scanning positions and scanning angles thereof; and
a tumor image positioning and storage module for recording at least one suspicious tumor ultrasound image as well as a scanning position and a scanning angles thereof, the tumor position and the maximum tumor dimension.

2. The breast ultrasound scanning aid system according to claim 1, further comprising a scanning trajectory module for recording a probe scanning trajectory and projecting said probe scanning trajectory onto a scanning trajectory map.

3. The breast ultrasound scanning aid system according to claim 2, further comprising a scanning failure alert module for analyzing the continuity of said probe scanning trajectory to determine whether to issue a scanning failure alert or not.

4. The breast ultrasound scanning aid system according to claim, further comprising a suspicious tumor alert module for issuing a suspicious tumor alert after identifying said at least one suspicious tumor ultrasound image from said breast ultrasound images.

5. The breast ultrasound scanning aid system according to claim 4, wherein said suspicious tumor alert module identifies at least one breast ultrasound image with a darker region from said breast ultrasound images and determines said at least one breast ultrasound image with said draker region as said suspicious tumor ultrasound image and said darker region as a suspicious tumor region.

6. The breast ultrasound scanning aid system according to claim 1, wherein said positioning module performs positioning magnetically or optically.

7. A breast ultrasound diagnosis aid system, comprising:
a representative image selection module for selecting at least one representative breast ultrasound image with tumor characteristics from a plurality of breast ultrasound images;
a tumor contour segmentation module for segmenting a suspicious tumor region from said representative breast ultrasound image; and
a computer-aided diagnosis module for acquiring a plurality of characteristic parameters in association with tumor tissues from said suspicious tumor region and providing a diagnostic suggestion after analyzing said characteristic parameters.

8. The breast ultrasound diagnosis aid system according to claim 7, further comprising an ultrasound mode analysis module for analyzing an ultrasound mode used in each of said breast ultrasound images.

9. The breast ultrasound diagnosis aid system according to claim 8, wherein said ultrasound mode is a B-mode, a Doppler mode or an elastography mode.

10. The breast ultrasound diagnosis aid system according to claim 8, further comprising an image selection module for providing a menu interface that displays each of said ultrasound mode used in each of said breast ultrasound images, wherein said breast ultrasound image with a specific ultrasound mode can be viewed through clicking said menu interface.

11. The breast ultrasound diagnosis aid system according to claim 7, wherein said representative image selection module for sorting said breast ultrasound images according to respective ratio values of darker regions or regions with harder elasticity in each of said breast ultrasound images and then selecting said representative breast ultrasound image from said sorted breast ultrasound images according to at least one image selection number acquired by multiplying the number of said breast ultrasound images with at least one image selection parameter.

12. The breast ultrasound diagnosis aid system according to claim 11, wherein said representative image selection module is provided with at least one color threshold value so that regions on said breast ultrasound images with average pixel values higher than said color threshold value are classified as lighter regions or regions with softer elasticity while regions on said breast ultrasound images with average pixel values lower than said color threshold value are classified as darker regions or regions with harder elasticity.

13. The breast ultrasound diagnosis aid system according to claim 7, wherein the elasticity of said suspicious tumor region is classified by a fuzzy c-means algorithm.

14. The breast ultrasound diagnosis aid system according to claim 7, wherein said tumor contour segmentation module identifies a tumor boundary of at least one suspicious tumor region from said representative breast ultrasound image, provides a plurality of control points on said tumor boundary of said suspicious tumor region so that said tumor contour segmentation module creates an re-formed tumor boundary of said suspicious tumor region by adjusting said control points.

15. The breast ultrasound diagnosis aid system according to claim 7, further comprising a diagnosis report generating module for receiving said diagnostic suggestion from said computer-aided diagnosis module so as to generate a breast diagnosis report.
